(19) United States
(12) Patent Application Publication
(10) Pub. No.: US 2017/0350109 A1
(43) Pub. Date: Dec. 7, 2017

(54) DEFUSE DEODORIZING DEVICE

(71) Applicant: Rashaun Gowdie, New York, NY (US)

(72) Inventor: Rashaun Gowdie, New York, NY (US)

(21) Appl. No.: 15/263,585

(22) Filed: Sep. 13, 2016

Related U.S. Application Data
(60) Provisional application No. 62/346,609, filed on Jun. 7, 2016.

(51) Int. Cl. E03D 9/00; A61L 9/013

(52) U.S. Cl. CPC E03D 9/005; A61L 9/013

(57) ABSTRACT

A deodorizing device for a toilet that includes: a housing; an oil container within the housing; an attachment means at the top of the housing; a container release point at the bottom of the oil container; tubing extending from the container release; and a release orifice at a distal end of the tubing. The attachment means may include hooks, where the hooks secure the housing onto a toilet tank. The oil container is preferably a capsule like device that is detachable from the housing. The deodorizing device may further include a toilet connector, where said toilet connector connects the tubing to the underside of a toilet seat.

This application claims priority to U.S. Provisional Application Ser. No. 62/346,609 filed on Jun. 7, 2016.

### BACKGROUND OF THE INVENTION

### Field of Invention

The present invention relates to a deodorizer used for a conventional toilet seat.

### Description of Related Art

Controlling odors in a bathroom due to toilet use is a common problem. Individuals use all types of approaches to address this issue. Hand held sprays may be used along with other deodorizers that may emit fragrance into the toilet bowl after and during use. Further room fresheners and fans are also used to diminish odors associated with toilet use. As a result, it would be advantageous to have further innovation to address the problem of odors emitting from a toilet after use.

### SUMMARY OF THE INVENTION

The present invention relates to a deodorizing device for a toilet that includes: a housing; an oil container within the housing; an attachment means at the top of the housing; a container release point at the bottom of the oil container; tubing extending from the container release; and a release orifice at a distal end of the tubing. The attachment means may include hooks, where the hooks secure the housing onto a toilet tank. The oil container is preferably a capsule like device that is detachable from the housing. The deodorizing device may further include a toilet connector, where said toilet connector connects the tubing to the underside of a toilet seat.

### BRIEF DESCRIPTION OF DRAWINGS

FIG. 1 depicts a deodorizing device for a toilet in accordance with the present invention.

### DETAILED DESCRIPTION

The present invention relates to a deodorizing device for a toilet that includes an oil container within a housing, where the housing attaches to a tank associated with a toilet bowl. A tubing extends from the oil container that releases oil into the toilet bowl providing a deodorizing effect during use. Preferably, eucalyptus oil is used within the oil container and droplets are released through a distal end of the tubing into the toilet bowl. As a result, a pleasant aroma is created along with the anti-bacterial solution to address the waste in the toilet bowl. The oil emitted into the toilet bowl masks the scent of any waste deposited by user. The oil therefore eliminates odors during toilet use and addresses unpleasant odors prior to their release into the air space. The oil effectively diminishes unpleasant odors of the waste and therefore prevents the problems before it is created. The release of oil into the toilet is controlled by a rubber button that when pushed forces the oil through the drip free pinhole nozzle. The rubber button controls the release of oil therefore a controlled amount is deposited into the toilet. An attachment is provided attaching the tubing to the toilet seat and securing the depositing end of the tube into the toilet bowl.

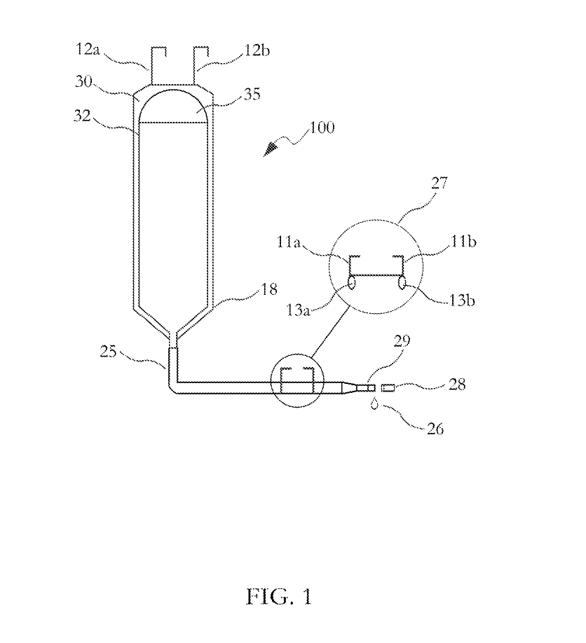

With reference to FIG. 1, a deodorizing device 100 in accordance with the present invention is depicted. The deodorizing device 100 includes a housing 30 with an oil container 32 within the housing 30. The oil container 32 is a capsule like device that is detachable from the housing 30 and replacable. Hooks 12a, 12b are provided at the top of the housing 30 to secure the housing 30 onto a toilet tank. A release 18 is provided at the bottom of the oil container 32 allowing gravity to release a portion of the oil as desired into the toilet. A release orifice 29 is provided at the distal end of a tube 25 that extends from container 32. Further shown in FIG. 1 is a toilet connector 27 that connects the tube 25 onto the underside of a toilet seat allowing for the tubing to release oil into the toilet bowl when the toilet seat is in a closed position. The connector 27 includes a bracket 11a, 11b and a rim braces 13a, 13b, where the bracket 11a, 11b secures the connector 27 to a toilet seat and the rim braces 13a, 13b engages the connector to a rim of a toilet bowl. The connector 27 is positioned on the tube 25 to insure that a pinhole nozzle 29 extends into the toilet bowl to allow emission of oil 26 into the toilet bowl. A cap 28 is also provided to seal the nozzle 29 as needed.

Each container 32 contains a determined amount of eucalyptus oil and includes a dropper 35, which includes a rubber button at the top of the container 32. The oil container 32 is secured inside housing 30. An air pocket is provided in the housing 30 so that once the button is pushed that air force is applied to the liquid and that is forced through the pinhole nozzle 29. Just like a medicine dropper, once the rubber button retracts to its normal position, air is pulled into the system creating a bubble that will immediately rise to the top of the liquid. This resets the system to its ready position.

The amount of oil that is dispersed into the water is controlled by the dimension of the rubber button. The dimension of the rubber button determines the amount of air that is applied to the surface of the liquid when pushed, which determines the amount that was forced through pinhole nozzle 29. The pinhole nozzle 29 is drip free. The plastic cap 28 is disposable and is intended to keep the oil secured in the container in event that rubber button was to be pushed pre-installation. The plastic cap 28 can be screwed off after or just before installation.

Preferably eucalyptus oil is used in the oil container. The oil has a unique property of providing an anti-bacterial solution with a pleasant solution therefore extinguishing unpleasant odor from waste deposited into the toilet bowl. Use of the present invention eliminates odors and effectively provides a pleasant aroma while using a toilet. The instant invention has been shown and described in what it considers to be the most practical and preferred embodiments. It is recognized, however, that departures may be made there from within the scope of the invention and that obvious modifications will occur to a person skilled in the art.

What is claimed is:

1. A deodorizing device for a toilet comprising:
a. a housing;
b. an oil container within the housing;
c. an attachment means at the top of the housing;
d. a container release point at the bottom of the oil container;
e. tubing extending from the container release; and
f. a release orifice at a distal end of the tubing.

2. The deodorizing device according to claim 1, where the attachment means includes hooks where the hooks secure the housing onto a toilet tank.

3. The deodorizing device according to claim 1, where the oil container is a capsule like device that is detachable from the housing.

4. The deodorizing device according to claim 1, further including a toilet connector, where said toilet connector connects the tubing to the underside of a toilet seat.

5. The deodorizing device according to claim 4, where the toilet connector includes a bracket and rim braces.

6. The deodorizing device according to claim 1, where the container includes dropper with a rubber button.

7. The deodorizing device according to claim 6, where rubber button controls the amount of oil released from the oil container.
